(19) United States
(12) Patent Application Publication
(10) Pub. No.: US 2024/0358620 A1
(43) Pub. Date: Oct. 31, 2024

(54) COSMETIC COMPOSITION WITH ENHANCED PHOTOPROTECTION PROPERTIES

(71) Applicant: SPECIALTY OPERATIONS FRANCE, LYON (FR)

(72) Inventors: Galder Cristobal, Singapore (SG); Haizhou Zhang, Shanghai (CN); Lynlie Lim, Singapore (SG)

(73) Assignee: SPECIALTY OPERATIONS FRANCE, LYON (FR)

(21) Appl. No.: 18/765,100

(22) Filed: Jul. 5, 2024

Related U.S. Application Data
(62) Division of application No. 17/416,797, filed on Jun. 21, 2021.

(51) Int. Cl. A61K 8/86; A61K 8/49; A61Q 17/04

(52) U.S. Cl. CPC A61K 8/86; A61K 8/4946; A61Q 17/04

(57) ABSTRACT

A cosmetic composition comprises a UV filter and a polymer for enhancing SPF of the composition. The use of said polymer is enhancing SPF of cosmetic composition.

### TECHNICAL FIELD

The present invention pertains to novel cosmetic compositions for photoprotection of the skin and/or the hair of human beings. Notably, the cosmetic composition comprises a UV filter and a polymer.

### BACKGROUND ART

Light radiation having wavelengths of from 280 nm to 400 nm, generally falling under the category of ultraviolet light radiation (also referred to as UV radiation), promotes tanning of human epidermis. For example, light radiation having wavelengths of from 280 to 320 nm, i.e., UV-B radiation, causes erythema and burning of the skin, which can impair the development of a natural tan. Therefore, it is highly desirable that such UV-B radiation is prevented from contacting the human skin or hair.

Light radiation having wavelengths from 320 to 400 nm, known as UV-A radiation, also adversely affects the human skin, in particular sensitive skin or skin which is continually exposed to solar radiation. UV-A radiation causes, in particular, a loss in elasticity of the skin and formation of wrinkles, thereby promotes a premature aging thereof. Such irradiation promotes triggering of the erythematous reaction or enhances this reaction in certain individuals, and may even be the source of phototoxic or photoallergic reactions. Thus, it is also desirable to screen UV-A radiation before it comes into contact with human skin.

A wide variety of cosmetic compositions for the photoprotection of human skin, notably for screening UV-A and/or UV-B radiation, are known in the art. These cosmetic compositions are typically oil-in-water emulsions which contain one or more lipophilic or hydrophilic compounds capable of selectively absorbing harmful or deleterious UV radiation. Such compounds are generally referred to as UL filters. They can be of organic or inorganic nature. The desired sun protection factor may be calculated based on these sunscreen compounds and the amounts thereof.

U.S. Pat. No. 5,607,664 teaches photoprotective/cosmetic compositions comprising, in a cosmetically acceptable vehicle, diluent or carrier that comprises a continuous aqueous phase, a mixture of (i) at least one photoprotective agent and (ii) at least one acrylic copolymer.

US 20080181858A1 teaches sunscreen compositions comprising one or more sunscreen agents, one or more film forming polymers, and a heat treated xanthan gum.

However, in order to achieve high SPF, the known cosmetic compositions generally require a high dosage of UV filter(s). There is a need to provide a cosmetic composition which can provide high SPF and at the same time, requires low dosage of UV filter to be included in the composition. There is a need to provide an agent which, when used in combination of a UL filter, can enhance the SPF of cosmetic compositions.

### SUMMARY OF INVENTION

In a first aspect, the present application provides a cosmetic composition comprising a UV filter and a polymer, wherein said polymer comprises:
a monomeric unit derived from a monomer represented by the general formula (I):

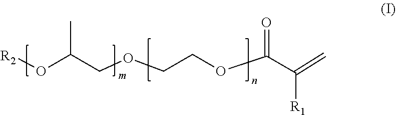

wherein R₁ represents H or a C₁-C₁₀ alkyl which is optionally substituted,
R₂ represents a C₂-C₃₀ alkyl which is optionally substituted or a bicycloheptenyl group,
m represents 0 or an integer of 1 to 100,
n represents an integer of 1 to 100;
a monomeric unit derived from a monomer selected from an ethylenically unsaturated carboxylic acid having 1 to 6 carbon atoms; a styrene-based compound, a vinyl ester and a vinyl ether; and
a monomer unit derived from an ester of ethylenically unsaturated carboxylic acid having 1 to 6 carbon atoms.

In a second aspect, the present application provides use of the polymer described herein for increasing SPF of a cosmetic composition containing a UV filter.

In a third aspect, the present application provides a method for enhancing SPF of a cosmetic composition containing a UV filter, comprising the step of adding the polymer described herein to said cosmetic composition.

### DETAILED DESCRIPTION

Throughout the description, including the claims, the term “comprising one” or “comprising a” should be understood as being synonymous with the term “comprising at least one”, unless otherwise specified, and “between” should be understood as being inclusive of the limits.

It should be noted that in specifying any range of concentration, weight ratio or amount, any particular upper concentration, weight ratio or amount can be associated with any particular lower concentration, weight ratio or amount, respectively.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

As used herein, the term “cosmetic composition” encompasses all compositions for the photoprotection of the skin and/or the hair against ultraviolet radiation.

As used herein, the term “UV filter” refers to a compound or a mixture of compounds, whether organic or inorganic, via the mechanisms of absorption and/or reflection and/or diffusion of UV-A and/or UV-B radiation, which prevents or reduces contact of a surface (notably the human skin and hair) with UV radiation, such as UV radiation from the sunlight and from artificial light sources. The UV filter is notably applied to the human skin or hair which is to be protected from the UV radiation.

As used herein, the terminology “Cn-Cm” in reference to an organic group, wherein n and m are each integers, indicates that the group may contain from n carbon atoms to m carbon atoms per group.

As used herein, the term “alkyl” means a saturated hydrocarbon radical, which may be straight or branched, such as methyl, ethyl, n-propyl, iso-propyl, n-butyl, sec-butyl, t-butyl, pentyl and n-hexyl.

As used herein, the term “cycloalkyl” means a saturated hydrocarbon radical that includes one or more cyclic alkyl rings, such as cyclopentyl, cyclooctyl, and adamantanyl.

As used herein, the term “alkoxyl” or “alkoxy” means an univalent group —RO (such as methoxyl) composed of an alkyl group united with oxygen.

As used herein, the term “hydroxyalkyl” means an alkyl radical, which is substituted with a hydroxyl groups, such as hydroxymethyl, hydroxyethyl, hydroxypropyl, and hydroxydecyl.

As used herein, the term “(meth)acrylate” means acrylate and/or methacrylate.

As used herein, the term “(meth)acrylic” means acrylic and/or methacrylic.

As used herein, the term “cosmetic composition” should be understood in a broad sense, which includes those compositions which are intended to be placed in contact with the external parts of the human body (e.g. skin, hair system, mucosa), exclusively or mainly to clean them, perfume them, change their appearance, protect them, keep them in good condition or correct body odors. In some instances, cosmetic compositions may also include health care compositions.

As used herein, “parts by weight” or “pbw” in reference to a named compound or material means the amount of the respective named compound or material, exclusive, for example, of any associated solvent.

As used herein, “optionally substituted” means that a radical group may be substituted by substitutent(s), or may not be substituted by substitutent(s). Said substitutent includes, but not limited to, alkyl, hydroxyl, halogen, alkoxyl, aryl, nitro, cyano and cycloalkyl.

As used herein, the term “polymer” refers to a macromolecule having a relatively high molecular mass that comprises chains of multiple repetitions of one or more monomeric units in which the monomeric units are derived, actually or conceptually, from molecules of relatively low molecular mass and are connected to form a linear, branched, or network structure. The polymer typically has a linear or branched structure, more typically single strand linear or branched structure, but may optionally be crosslinked. The monomeric units of the polymer may be arranged in any sequence, including random, alternating, tapered, or block sequence, along the polymer chain. In context of the present invention, polymer includes copolymers.

The present application provides a cosmetic composition comprising a UV filter and a polymer, wherein said polymer comprises:
(a) a monomeric unit derived from a monomer represented by the general formula (I):

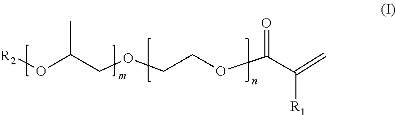

wherein R₁ represents H or a C₁-C₁₀ alkyl which is optionally substituted,
R₂ represents a C₂-C₃₀ alkyl which is optionally substituted or a bicycloheptenyl group,
m represents 0 or an integer of 1 to 100,
n represents an integer of 1 to 100;
(b) a monomeric unit derived from a monomer selected from a ethylenically unsaturated carboxylic acid having 1 to 6 carbon atoms; a styrene-based compound, a vinyl ester, a vinyl ether and mixture thereof; and
(c) a monomer unit derived from an ester of ethylenically unsaturated carboxylic acid having 1 to 6 carbon atoms.

According to the invention, the polymer comprises:
(a) a monomeric unit derived from a monomer represented by the general formula (I):

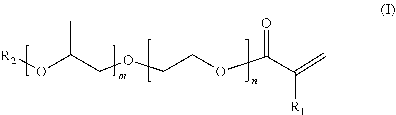

wherein R₁ represents H or a C₁-C₁₀ alkyl which is optionally substituted,
R₂ represents a C₂-C₃₀ alkyl which is optionally substituted or a bicycloheptenyl group,
m represents 0 or an integer of 1 to 100,
n represents an integer of 1 to 100;
(b) a monomeric unit derived from a monomer selected from an ethylenically unsaturated carboxylic acid having 1 to 6 carbon atoms; styrene-based compounds, vinyl ester, vinyl ether and mixture thereof, and
(c) a monomer unit derived from a C₁-C₁₀ alkyl ester of ethylenically unsaturated carboxylic acid having 1 to 6 carbon atoms.

Preferably, R₁ as defined in general formula (I) represents C₁-C₆ alkyl, more preferably, C₁-C₄ alkyl, and most preferably methyl or ethyl.

In some embodiments, R₂ as defined in general formula (I) represents a C₂-C₃₀ alkyl, preferably a C₁₀-C₃₀ alkyl, and even more preferably, a C₁₆-C₂₂ alkyl. For example, R₂ may be tridecyl, tetradecyl, pentadecyl, hexadecyl, heptadecyl, octadecyl, nonadecyl, eicosyl, heneicosyl, behenyl, tricosyl, tetracosyl, pentacosyl, hexacosyl, heptacosyl, octacosyl, nonacosyl, or triacontyl, more preferably, hexadecyl, heptadecyl, octadecyl, nonadecyl, eicosyl, or behenyl.

It is appreciated that each hydrogen in R₁ and/or R₂ may be substituted, for example, substituted by a hydroxyl or halogen moiety.

In some embodiments, R₂ as defined in formula (I) represents a bicycloheptenyl group. Said bicycloheptenyl group may be derived from, for example, terpenic compounds having a core (non-substituted) 7 carbon atom bicyclic ring systems according to any one of (II) to (V.b):

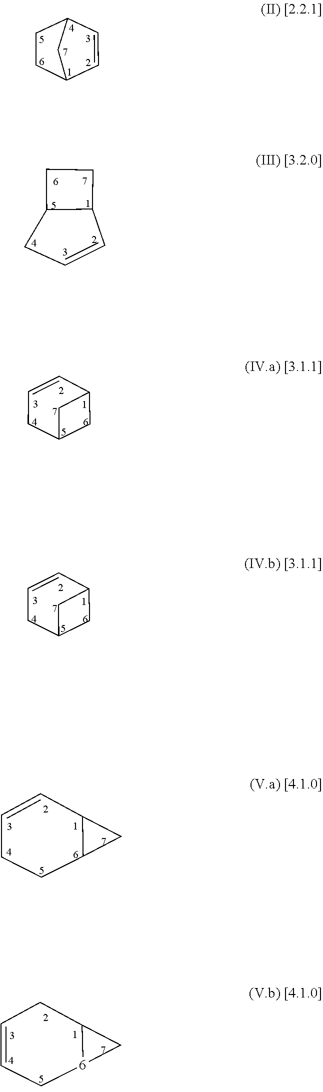

For instance, R₂ as defined in formula (I) may be a bicyclo[d.e.f]heptenyl wherein d is 2, 3, or 4, e is 1 or 2, f is 0 or 1, and the sum of d+e+f=5, and which may, optionally, be substituted on one or more of the ring carbon atoms by one or more (C₁-C₆) alkyl groups. For instance, R₂ as defined in formula (I) may be:
a bicyclo[2.2.1]heptenyl group that is bonded to other moeity of formula (I), via its carbon atom at the 2-position or 3-position and is typically substituted on its carbon atom at the 7 position by one or two (C₁-C₆) alkyl radicals, more typically by two methyl radicals, or
a bicyclo[3.1.1]heptenyl group that is bonded to other moiety of formula (I), via its carbon atom at the 2-position or 3-position and is typically substituted on its carbon atom at the 6-position or 7-position by one or two (C₁-C₆) alkyl radicals, more typically by two methyl radicals.

In a preferred embodiment, R₂ as defined in formula (I) is selected from:

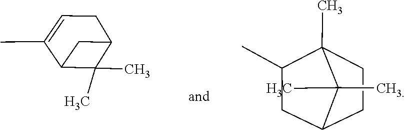

In general formula (I), m represents 0, or an integer of 1 to 100, preferably an integer of 1 to 50, more preferably an integer of 1 to 20, even more preferably an integer of 1 to 15, and most preferably an integer of 3 to 10. n represents an integer of 1 to 100, preferably an integer of 5 to 80, more preferably an integer of 10 to 50, even more preferably an integer of 15 to 40, and most preferably an integer of 20 to 30.

In particular, the polymer comprises, as the monomeric unit (a) component:
(a1) a monomeric unit derived from a monomer represented by the general formula (VI):

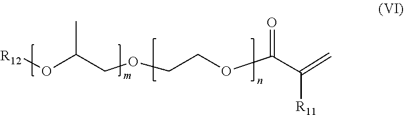

wherein R₁₁ represents H or a C₁-C₁₀ alkyl,
R₁₂ represents a C₂-C₃₀ alkyl,
m represents 0 or an integer of 1 to 100,
n represents an integer of 1 to 100;
(a2) a monomeric unit derived from a monomer represented by the following formula (VII):

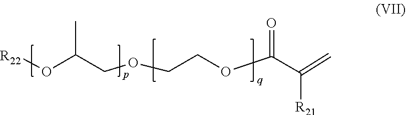

wherein R₂₁ represents H or C₁-C₁₀ alkyl,
R₂₂ represents a bicycloheptenyl group,
p represents 0 or an integer of 1 to 100,
q represents an integer of 1 to 100.

Typically, R₁₁ as defined in formula (VI) and R₂₁ as defined in formula (VII) may be those as defined as R₁ group in formula (I), and R₁₂ as defined in formula (VI) and R₂₂ as defined in formula (VII) may be those defined as R₂ group in formula (I).

In general formula (VI), m represents 0, or an integer of 1 to 100, n represents an integer of 1 to 100. Preferably, m is 0 and n is an integer of 1 to 50. More preferably, m is 0 and n is an integer of 10 to 40.

In general formula (VII), p represents 0, or an integer of 1 to 100, q represents an integer of 1 to 100. Preferably, p is an integer of 1 to 10 and q is an integer of 1 to 50.

For monomeric unit (b), said monomer may be selected from ethenylically unsaturated carboxylic acids having 1 to 6 carbon atoms, such as acrylic acid and methacrylic acid, ethylenically unsaturated dicarboxylic acids having 1 to 6 carbon atoms, such as maleic acid and fumaric acid; styrenes; substituted styrenes, such as methyl styrene, ethyl styrene and propyl styrene; vinyl esters, such as vinyl acetate, vinyl propionate and vinyl 2-ethylhexanoate; vinyl ethers, such as methyl vinyl ether, ethyl vinyl ether, butyl vinyl ether and hydroxybutyl vinyl ether; and mixture thereof.

Suitable (meth)acrylic acid includes, for example, non-substituted (meth)acrylic acid and (meth)acrylic acid substituted by C₁-C₆ alkyl group, hydroxyl group and/or halogen. Typically, said monomer is selected from the group consisting of acrylic acid, methacrylic acid, and mixtures thereof.

For monomeric unit (c), said monomer may be selected from an ester of ethenylically unsaturated carboxylic acid having 1 to 6 carbon atoms (such as acrylic acid and methacrylic acid), a mono- or diester of ethylenically unsaturated dicarboxylic acid having 1 to 6 carbon atoms (such as maleic acid and fumaric acid), and mixtures thereof. Typically, said monomer is selected from an ester of (meth)acrylate and mixture thereof. Said ester of (meth)acrylate may be C₁-C₂₂ alkyl ester of (meth)acrylate, C₁-C₂₂ hydroxyalkyl ester of (meth)acrylate, C₂-C₂₂ alkoxyalkyl ester of (meth)acrylate, C₆-C₂₄ cycloalkyl ester of (meth)acrylate, (C₆-C₄₀) aryl ester of (meth)acrylate, (C₇-C₄₀) aralkyl ester of (meth)acrylate, and mixture thereof. Preferably, said monomeric unit (c) is C₁-C₂₂ alkyl ester of (meth)acrylate, more typically, C₁-C₁₀ alkyl ester of (meth)acrylate, even more typically C₁-C₆ alkyl ester of (meth)acrylate, and still more typically C₁-C₄ alkyl ester of (meth)acrylate.

Suitable esters of (meth)acrylate include, for example, methyl (meth)acrylate, ethyl (meth)acrylate, butyl (meth)acrylate, isobutyl (meth)acrylate, cyclohexyl (meth)acrylate, 2-ethylhexyl (meth)acrylate, isodecyl (meth)acrylate, lauryl (meth)acrylate isobornyl (meth)acrylate, benzyl (meth)acrylate, hydroxyethyl (meth)acrylate, hydroxypropyl (meth)acrylate, methoxyethyl (meth)acrylate, ethoxyethyl (meth)acrylate, phenoxyethyl (meth)acrylate, tetrahydrofurfuryl (meth)acrylate, glycidyl (meth)acrylate, dimethylaminoethyl (meth)acrylate, diethylaminoethyl (meth)acrylate, tert-butylaminoethyl (meth)acrylate, and acetoxyethyl (meth)acrylate.

According to every one of the invention embodiments, the polymer preferably comprises:
(a1) a monomeric unit derived from a monomer represented by the general formula (VI):

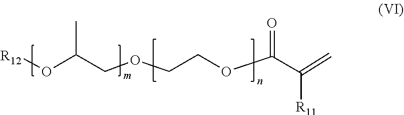

wherein R₁₁ represents H or a C₁-C₁₀ alkyl,
R₁₂ represents a C₂-C₃₀ alkyl,
m represents 0 or an integer of 1 to 100,
n represents an integer of 1 to 100;
(a2) a monomeric unit derived from a monomer represented by the following formula (1):

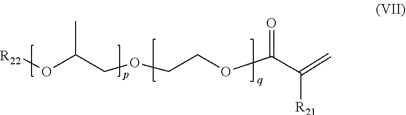

wherein R₂₁ represents H or C₁-C₁₀ alkyl,
R₂₂ represents a bicycloheptenyl group,
p represents 0 or an integer of 1 to 100,
q represents an integer of 1 to 100;
(b) a monomeric unit derived from a monomer selected from ethylenically unsaturated carboxylic acid having 1 to 6 carbon atoms; styrene-based compounds and vinyl acetate; and
(c) a monomer unit derived from C₁-C₁₀ alkyl ester of ethylenically unsaturated carboxylic acid having 1 to 6 carbon atoms.

The polymer suitable for the present invention may further comprise monomeric units derived from an additional monomer. Examples of the additional monomer include and are not limited to: ethylenically unsaturated monomers including (meth)acrylamides such as, (meth)acrylamide, N-methylol (meth)acrylamide, N-butoxyethyl (meth)acrylamide, N,N-dimethyl (meth)acrylamide, N-isopropyl (meth)acrylamide, N-tert-butyl (meth)acrylamide, N-tert-octyl (meth)acrylamide, and diacetone (meth)acrylamide; and N-vinylamides such as N-vinylpyrrolidione, N-vinylcaprolactam, N-vinylformamide and N-vinylacetamide.

The polymer advantageously comprises 4-15% by weight of monomeric unit (a), typically 8-14% by weight of monomeric unit (a), based on the weight of the polymer.

The polymer advantageously comprises 30-50% by weight of monomeric unit (b), typically 35-45% by weight of monomeric unit (b), based on the weight of the polymer.

The polymer advantageously comprises 30-70% by weight of monomeric unit (c), typically 40-60% by weight of monomeric unit (c), based on the weight of the polymer.

According to every one of the invention embodiments, with respect to the monomeric unit (a1), it may be comprised in amount of 1-10% by weight of the polymer, more typically, it may be comprised in amount of 2-8% by weight of the polymer; with respect to the monomeric (a2), it may be comprised in amount of 1-10% by weight of the polymer, more typically 2-6% by weight of the polymer.

According to every one of the invention embodiments, the polymer may comprise 0-20% by weight of monomeric units derived from by additional monomer(s), more typically 0.1-15% by weight, and even more typically 1-10% by weight, based on the weight of the polymer.

The polymer suitable for the present invention may be prepared by a polymerization process known by a skilled person. For example, the polymer may be prepared by the processes disclosed in WO 2011100071A1.

Generally, the polymer is comprised in amount of 1-20% by weight of the cosmetic composition, more typically 1-15% by weight of the composition, even more typically 2-10% by weight of the composition, and most typically 3-8% by weight of the composition.

The UV filter advantageously includes, for example, a UV-absorbing organic UV filter, an inorganic (nano) pigment or a mixture thereof.

The organic UV filter according to the present application includes, for example, one or more conventional hydrophilic or lipophilic organic sunscreen agents (absorbing agents) which are active in the UV-A and/or UV-B region. Exemplary of such organic UV filter are 2-phenylbenzimidazole-5-sulfonic acid and salts thereof, cinnamic derivatives such as, for example, 2-ethylhexyl p-methoxycinnamate, salicylic derivatives such as, for example, 2-ethylhexyl salicylate and homomenthyl salicylate, camphor derivatives such as, for example, 3-(4-methylbenzylidene)camphor or (1,4-divinylbenzene)camphorsulfinic acid, triazine derivatives such as 2,4,6-tris[p-(2′-ethylhexyl-1′-oxycarbonyl)anilino]-1,3,5-triazine, benzophenone derivatives such as 2-hydroxy-4-methoxybenzophenone, dibenzoylmethane derivatives such as 4-tert-butyl-4′-methoxydibenzoylmethane, .beta., .beta.-diphenylacrylate derivatives such as 2-ethylhexyl .alpha.-cyano-.beta., .beta.-diphenylacrylate, p-aminobenzoic acid derivatives such as, for example, octyl and para-dimethylaminobenzoate, menthyl anthranilate and the sunscreen polymers and sunscreen silicones described in WO-93/04,665. Other examples of organic sunscreen compounds are described in EP-A-0,487,404.

The metal oxides constituting the inorganic (nano) pigments suitable for formulation into the cosmetic compositions according to the present invention comprise those which are per se known for their photoprotective activity. Thus, they are advantageously selected from among titanium, zinc, iron, zirconium and cerium oxides, or mixtures thereof. Preferably, nanopigments of metal oxides are employed.

Such nanopigments of metal oxides, whether coated or uncoated, are materials known to this art and described, in particular in EP-A-0,518,773, hereby expressly incorporated by reference. Additional and commercially available nanopigments not described therein, but which are also suitable according to this invention include the products marketed under the trademarks UVT M 160, UVT M 212 and UVT M 262 by Kemira, and MT 100 SA and MT 100 SAS by Tayca.

In a preferred embodiment of the invention, inorganic nanopigments are employed that are based on titanium dioxide. This titanium dioxide may be in a crystallized state of rutile and/or anatase type, and/or in an amorphous or substantially amorphous form. As indicated above, this pigment either may or may not then be coated, but it is preferable to use coated pigments, for example coated with alumina and/or aluminum stearate.

Generally, the UV filter is comprised in amount of 1-30% by weight of the composition, more typically 2-25% by weight of the composition, even more typically 5-20% by weight of the composition, and most typically 5-15% by weight of the composition.

Photoprotection properties of a cosmetic composition can typically be represented by the level of Sun Protection Factor (SPF). SPF is expressed mathematically by the ratio of the irradiation time required to attain the erythema-forming threshold with the cosmetic composition to the time required to attain the erythema-forming threshold in absence of the cosmetic composition. SPF can be measured by in vitro methods or in vivo methods which are known by a skilled person.

Without wishing to be bound by theory, it is believed that in the composition of the invention, the polymer described herein increases the SPF of the composition, and such enhancement in SPF requires low dosage of UV filter being added in the composition. For example, the polymer described herein increases the SPF of the composition by more than 3 times, typically 4 times or more, compared to the calculated SPF of compositions having the UV filter alone.

The compositions of the present invention may additionally comprise conventional cosmetic additives and adjuvants selected especially from fats, organic solvents, ionic or nonionic thickening agents, softeners, antioxidants and especially anti-free-radical antioxidants, opacifying agents, stabilizing agents, emollients, silicones, .alpha.-hydroxy acids, anti-foaming agents, hydrating agents, vitamins, fragrances, preservatives, surfactants, fillers, insect repellants, sequestering agents, polymers, 0 propellants, basifying or acidifying agents, dyes and colorants, or any other ingredient usually employed in cosmetics, in particular for the production of sunscreen/cosmetic compositions in the form of oil-in-water emulsions.

The cosmetic compositions according to the present invention can optionally further include agents having biological activities. Such agents include, for example, anti-acne agents, antimicrobial agents, anti-inflammatory agents, analgesics, anti-erythemal agents, antiruritic agents, antiedermal agents, antipsoriatic agents, antifungal agents, skin protectants, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, hydroxyalkyl urea, amino acids, peptides, minerals, ceramides, biohyaluronic acids, vitamins, skin lightening agents, self tanning agents, coenzyme Q10, niacinimide, capcasin, caffeine, and any combination of any of the foregoing.

The compositions of the invention may be formulated according to techniques well known by a skilled person in particular those intended for the preparation of emulsions of oil-in-water type.

The present invention will be further illustrated with reference to the following examples.

### EXAMPLES

### Materials

| Ingredients | Function | Supplier |
|---|---|---|
| Deionized Water | — | |
| Sodium Hydroxide 20% solution | pH adjuster | |
| Isohexadecane | Emollient | |
| Mackaderm OC (Ethyhexyl Cocoate) | Emollient | Solvay |
| | | Company |
| Neo Heliopan AV (Ethylhexyl | UV Filter | Symrise |
| Methoxycinnamate) | | Company |
| Neo Heliopan 357 (Butyl Methoxydebenzoyl | UV filter | Symrise |
| Methane) | | Company |
| Neo Heliopan Hydro (Phenylbenzimidazole | UV filter | Symrise |
| Sulfonic Acid) 30% solution | | Company |

### Method of Determining In Vitro SPF

The Sun Protection Factor (SPF) of a sample was determined using the in vitro technique described by B. L. Diffey et a1, in J. Soc. Cosmet. Chem., 40, 127-133 (1989). This technique entailed determining the monochromatic protection factors every 5 nm over a wavelength range of from 290 to 400 nm, and in calculating the sun protection factor from these factors according to a given mathematical equation.

### Example 1

Water and a sulfated alcohol ethoxylate (Rhodapex AB20, Solvay) were charged to a reaction vessel and heated to about 65° C., while purging with N₂. A N₂ blanket was maintained throughout each run. When the temperature reached about 65° C., a 25% of Initiator solution and 2% of monomer emulsion were added to the reaction vessel. The temperature was then maintained at about 65° C. for about 15 minutes. The remaining monomer emulsion and initiator solution were fed into the reaction vessel at a steady rate over 3 hours. Once the monomer and initiator feeds were completed, the contents of the reaction vessel were maintained at about 65° C. for about 1 hour and then a chaser solution consisting of t-butylperoxy benzoate added to the reaction vessel in one shot, followed by continuous addition of erythorbic acid solution over 30 minutes, was introduced to the reaction vessel. Once introduction of the chaser solution was completed, the contents of the reaction vessel were maintained at about 65° C. for 90 minutes, and then allowed to cool.

The ingredients used are summarized in TABLE 1 below. The polymer of Example 1 contained:
a. NOPOL polyether monomer according to the general formula (I) wherein R₁=methyl, m=5, n=25, R₂=

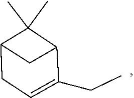

4.7 wt %
b. a C₁₆-C₂₂ alkyl-polyethoxylated methacrylate according to the general formula (I), wherein R₁=methyl, m=0, n=25, R₂=C₁₆-C₂₂ alkyl (“C₁₆-C₂₂ alkyl-polyether monomer”), 6.6 wt %
c. methacrylic acid, 38.7 wt %
d. ethyl acrylate, 50.0 wt %.

The average particle size as determined by light scattering, of the resultant polymer of Example 1 was about 103 nm.

The NOPOL polyether monomer was introduced in the form of an aqueous solution (“NOPOL polyether monomer solution”) that contained, based on 100 pbw of the solution, about 50 pbw of the NOPOL polyether monomer and about 25 pbw MAA. The C₁₆-C₂₂ alkyl-polyether monomer was introduced in the form of an aqueous solution (“C₁₆-C₂₂ alkyl-polyether solution”) that contained, based on 100 pbw of the solution, about 50 pbw of the C₁₆-C₂₂ alkyl-polyether monomer and about 25 pbw MAA.

TABLE 1

| | Charges(g) |
|---|---|
| Kettle charge | |
| Water | 323.9 |
| Rhodapex AB20 | 2.07 |
| (sulfated alcohol ethoxylate, 29% solids content) | |
| Monomer solution | |
| Water | 300 |
| Rhodapex AB20 | 20.7 |
| (sulfated alcohol ethoxylate, 29% solids content) | |
| Ethyl acrylate (EA) | 159.0 |
| Methacrylic acid (MAA) | 111.0 |
| NOPOL polyether monomer solution | 24.0 |
| C₁₆-C₂₂ alkyl-polyether solution | 36.0 |
| Initiator solution | |
| Ammonium persulfate | 0.84 |
| Water | 79.7 |
| Chaser solution | |
| Part 1: t-butylperoxybenzoate | 0.60 |
| Part 2: | |
| Water | 19.7 |
| Erythorbic acid | 0.30 |
| Total | 1077.8 |

### Examples 2-10

The latex polymer obtained in Example 1 above was added in water 5 with mixing at 200 rpm (Eurostar 60 digital, IKA) for 10 min. Triethanolmine was added to the pH to 6.8. Phase A was thus obtained. Phase B as identified in Table 2 below was added into the Phase A above with continuous mixing at 1000 rpm (Eurostar 60 digital, IKA) for 10 min, until all homogenous. Phase C as identified in TABLE 2 was gradually into 10 above system with the agitation at 200 rm (Eurostar 60 digital, IKA) for 10 min. Phase D as identified in Table 2 was into above system with the agitation at 200 rm (Eurostar 60 digital, IKA) for 10 min. Then, the formulations as identified in TABLE 2 below were obtained.

TABLE 2

| Example No. | | 2 | 3 | 4 | 5 | 6 |
|---|---|---|---|---|---|---|
| Phase A | Deionized Water | QS | QS | QS | QS | QS |
| | Latex polymer | / | 6.0% | 6.0% | 6.0% | 6.0% |
| | obtained in | | | | | |
| | Example 1 | | | | | |
| | Triethanolmine | QS | QS | QS | QS | QS |
| Phase B | Isohexadecane | 4.0% | 4.0% | 4.0% | 4.0% | 4.0% |
| | Mackaderm OC | 6.0% | 6.0% | 6.0% | 6.0% | 6.0% |
| | Neo Heliopan AV | 6.0% | 6.0% | 5.4% | 4.8% | 4.2% |
| | Neo Heliopan 357 | 4.0% | 4.0% | 3.6% | 3.2% | 2.8% |
| Phase C | Neo Heliopan Hydro | 10.0% | 10.0% | 9% | 8% | 7% |

| Example No. | | 7 | 8 | 9 | 10 |
|---|---|---|---|---|---|
| Phase A | Deionized Water | QS | QS | QS | QS |
| | Latex polymer | 5.0% | 5.0% | 5.0% | 5.0% |
| | obtained in | | | | |
| | Example 1 | | | | |
| | Triethanolmine | QS | QS | QS | QS |
| Phase B | Isohexadecane | 4.0% | 4.0% | 4.0% | 4.0% |
| | Mackaderm OC | 6.0% | 6.0% | 6.0% | 6.0% |
| | Neo Heliopan AV | 6.0% | 5.4% | 4.8% | 4.2% |
| | Neo Heliopan 357 | 4.0% | 3.6% | 3.2% | 2.8% |
| Phase C | Neo Heliopan Hydro | 10.0% | 9% | 8% | 7% |

### QS: Suitable Amount

The in vitro SPFs of Examples 2-10 were determined and the results were listed in TABLE 3 below:

TABLE 3

| | 2 | 3 | 4 | 5 | 6 | 7 | 8 | 9 | 10 |
|---|---|---|---|---|---|---|---|---|---|
| In vitro | 20 | 148.03 | 63.73 | 54.15 | 51.4 | 89.5 | 60.47 | 31.97 | 25.87 |
| SPF | | | | | | | | | |

1. (canceled)

2. The method according to claim 15, wherein R₂ as defined in general formula (I) represents a bicycloheptenyl group derived from a terpenic compound having a core (non-substituted) 7 carbon atom bicyclic ring system according to any one of (II) to (V.b):

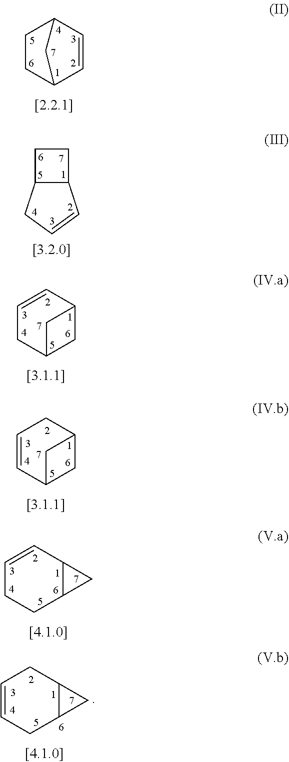

3. The method according to claim 15, wherein m represents an integer of 1 to 50 and n represents an integer of 5 to 80.

4. The method according to claim 15, wherein the content of said monomeric unit (a) is 4 to 15 wt %, the content of said monomeric unit (b) is 30 to 50 wt %, and the content of said monomeric unit (c) is 30 to 70 wt %, based on total weight of the polymer.

5. (canceled)

6. The method according to claim 16, wherein R₂₂ as defined in general formula (VII) represents a bicycloheptenyl group derived from a terpenic compound having a core (non-substituted) 7 carbon atom bicyclic ring system according to any one of (II) to (V.b):

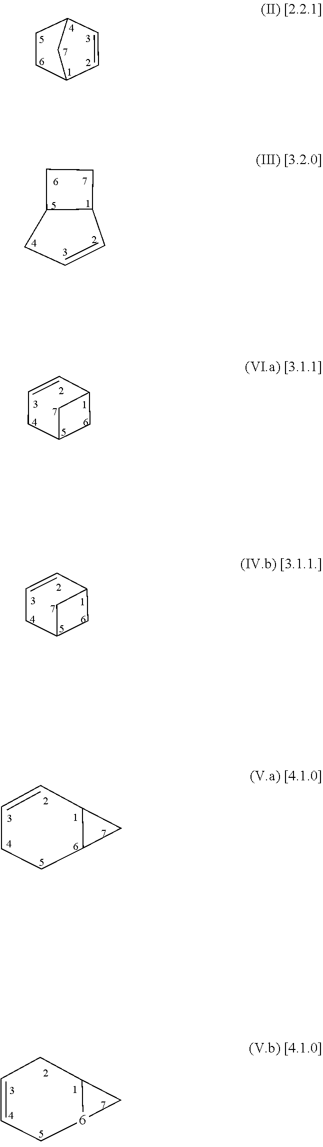

7. The method according to claim 16, wherein R₂₂ as defined in general formula (VII) is a bicyclo[d.e.f]heptenyl, wherein: d is 2, 3, or 4, e is 1 or 2, f is 0 or 1, the sum of d+e+f=5, and each ring carbon atom is optionally substituted with a C₁-C₆ alkyl group.

8. The method according to claim 16, wherein R₂₂ as defined in general formula (VII) is selected from:

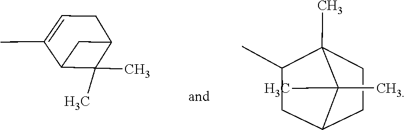

9. The method according to claim 16, wherein m represents an integer of 1-50, n represents an integer of 5-80, p represents an integer of 1-50, and q represents an integer of 5-80.

10. The method according to claim 15, wherein said monomeric unit (b) is derived from a (meth)acrylic acid.

11. The method according to claim 15, wherein said monomeric unit (c) is derived from a C₁ to C₁₀ alkyl ester of (meth)acrylate.

12. The method according to claim 15, wherein the UV filter comprises a UV-absorbing organic UV filter, an inorganic (nano) pigment or a mixture thereof.

13. (canceled)

14. (canceled)

15. A method for enhancing SPF of a cosmetic composition containing a UV filter, comprising the step of adding a polymer to said cosmetic composition; wherein said polymer comprises:
a) a monomeric unit derived from a monomer represented by the general formula (I): 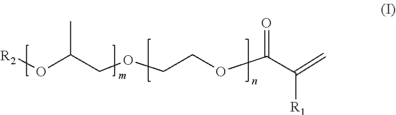
wherein R₁ represents H or a C₁-C₁₀ alkyl which is optionally substituted,
R₂ represents a C₂-C₃₀ alkyl which is optionally substituted or a bicycloheptenyl group,
m represents 0 or an integer of 1 to 100, and
n represents an integer of 1 to 100;
b) a monomeric unit derived from a monomer selected from an ethylenically unsaturated carboxylic acid having 1 to 6 carbon atoms; a styrene-based compound and a vinyl acetate; and
c) a monomer unit derived from a C₁-C₁₀ alkyl ester of ethylenically unsaturated carboxylic acid having 1 to 6 carbon atoms.

16. The method of claim 15, wherein said monomeric unit (a) comprises:
(a1) a monomeric unit derived from a monomer represented by the general formula (VI): 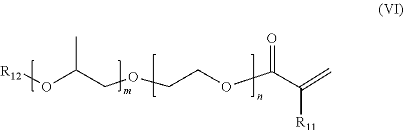
wherein R₁₁ represents H or a C₁-C₁₀ alkyl which is optionally substituted,
R₁₂ represents a C₂-C₃₀ alkyl which is optionally substituted,
m represents 0 or an integer of 1 to 100, and
n represents an integer of 1 to 100;
(a2) a monomeric unit derived from a monomer represented by the following formula (VII): 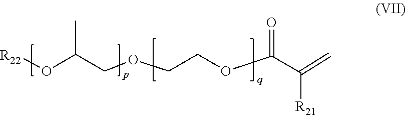
wherein R₂₁ represents H or a C₁-C₁₀ alkyl which is optionally substituted,
R₂₂ represents a bicycloheptenyl group,
p represents 0 or an integer of 1 to 100, and
q represents an integer of 1 to 100.
